(19) United States
(12) Patent Application Publication
(10) Pub. No.: US 2026/0269082 A1
(43) Pub. Date: Sep. 10, 2026

(54) ENVIRONMENTAL PRESSURE OF INFECTION

(71) Applicants: EUDIKA S.A., Pando, Canelones (UY); METABIX BIOTECH LLC, Wilmington, DE (US)

(72) Inventors: Laura Maccio, Pando, Canelones (UY); Andres Pio, Pando, Canelones (UY); Martin Masner, Pando, Canelones (UY)

(73) Assignees: EUDIKA S.A., Pando, Canelones (UY); METABIX BIOTECH LLC, Wilmington, DE (US)

(21) Appl. No.: 18/875,556

(22) PCT Filed: Apr. 17, 2024
(86) PCT No.: PCT/US2024/024905

§ 371 Date: Dec. 16, 2024

(30) Foreign Application Priority Data
Aug. 24, 2023 (UY) 40404

(51) Int. Cl. G16H 50/80; G16H 10/40

(52) U.S. Cl. CPC G16H 50/80; G16H 10/40

(57) ABSTRACT

A method for disease risk prediction in epidemiological units of production animals by determining Environmental Pressure of Infection (EPI). Determining EPI comprises establishment of threshold Environmental Pressure of Infection (EPIt), construction of Environmental Baseline (EBL), and generation of disease risk alerts.

### CROSS-REFERENCE TO RELATED APPLICATIONS

The present application claims priority to International Patent Application No. PCT/US24/24905, filed Apr. 17, 2024, which claims priority to Uruguay Patent Application No. 40404, filed Aug. 24, 2023, the entire disclosure of which is incorporated by reference into the present application.

### FIELD OF INVENTION

The present invention is related to the field of animal health and the generation of alerts arising from the detection of Environmental Pressure of Infection in epidemiological units of production animals using a variety of molecular and epizootiological techniques.

### BACKGROUND

Air pollution represents a high environmental risk for humans and animals, since the air contains inorganic contamination together with ABPs (Airborne Biological Particles) such as bacteria, viruses, fungi, among others, that come from different parts of the environment, such as soils, plants, and water.

The probability that an animal becomes ill, and its severity depends on the infectious potential of the pathogen and its aerosolization, among other factors. In animal husbandry environments, such as livestock facilities, exposure to bacteria, viruses, fungi, and parasites can have a significant impact on animal health, production efficiency, and ultimately, public health.

Currently, the problem of diseases caused by viruses and bacteria are addressed with some of the following tools: preventive plans with drugs, for example, vaccines and antibiotics; sampling for diagnosis by necropsy and bleeding when there are suspicions of diseases; and control of the vaccination plan.

The use of drugs, in many cases is necessary and irreplaceable, as can be some specific vaccines, however, there are no efficient vaccines for all known and potential pathogens. On the other hand, the indiscriminate use of antimicrobials leads to the generation of antimicrobial resistance, which is a potential problem for animal and human health. The method proposed in the present application has two advantages over this: it can be adapted to detect any emerging or new pathogen that appears in nature, and, on the other hand, it allows detecting biological threats at the moment they appear, allowing adequate treatment and resulting in reducing the unnecessary use of antibiotics.

Sampling by necropsy, collection of saliva, cloaca, or bleeding upon suspected diseases has the main disadvantage that it is carried out once the disease has progressed, that is, there are symptoms or mortality, thus not solving the anticipation problem. In addition to this, there are biological agents that cause death in animals, but their traces in tissues do not last long enough to be detected later, such as avian metapneumovirus. Therefore, in the present invention it is proposed to carry out sampling plans that allow the detection of the causative biological agents in advance of the manifestation of the disease.

Conventional methods used to detect infections in animals may be prone to bias and/or limitations in terms of the representativeness or diversity of pathogens detected, as well as in terms of the time these methods require to give results. In direct detection of pathogens, classical microbiology techniques such as bacteriological cultures have been used, which are simple and useful, but have days or weeks of delay in their result (mycoplasma case), or impossibility of application due to the non-cultivable universe of microorganisms, which is much greater than cultivable one. A serological test such as ELISA, which is carried out on serum samples from individuals and has an antibody-antigen detection, can be too laborious or have a low precision in the detection of pathogens in samples, since it can be difficult with the presence of other microorganisms and even more if there is a low concentration of the target pathogen (Hosseini et. al, 2018) as it is at the beginning of an infection in a population.

Regarding the control of the vaccination plan, carried out by ELISA, this follow-up involves sampling methods that are frequently invasive (in some cases they involve the sacrifice of the animals and taking blood samples), laborious, and may not be very representative, especially at the beginning of an infection. The method presented in this application is not invasive, it is easy to implement, and it is representative of the sampled environment.

Additionally, coupled detection methods such as ELISA by serology have disadvantages, such as having to wait for the immune response to occur with the pathogen, and other conditions. The advantages and novelty of the method described in the present invention compared to ELISA are shown compared to the present method summarized in the table shown in FIG. 1.

Other techniques have been used to detect pathogens in a sample such as PCR and next-generation sequencing (NGS), which have been shown to provide rapid, comprehensive, and accurate detection of pathogens in environmental and clinical samples, using PCR for amplification of nucleic acids for specific genes and the detection of specific sequences by means of fluorescent probes or hybridization assays or identification by qPCR as stated in patent application WO2021250274A1. On the other hand, NGS allows us to do massive sequencing of DNA and/or RNA fragments much faster and less expensively than the Sanger sequencing method, used long before NGS, as indicated in U.S. Pat. No. 11,485,969 B2.

Current methods for sampling and subsequent detection of pathogens in the environment may be limited in terms of sensitivity, efficiency, and logistics. Sample transport requires specific transport solutions, and the stability of pathogens in these solutions can be affected by water activity and other factors. Various aerial sampling devices have been used to study the communities. The Hirst-type device has been a good option for monitoring communities, but it has not been able to take viruses into account, so it is not complete, besides being expensive and not portable. Polytetrafluoroethylene filters have also been good, but there is a lack of an appropriate analytical method for the entire aerial microbiota sample (WO2021250274A1). Added to this, none of these methods by themselves provide alerts or reports that allow producers to anticipate the negative impacts of pathogens present in the environment.

To determine the presence of pathogens in the air, there are a series of devices and methods to capture and analyze airborne organisms such as those reported in patent applications WO2021250274A1, WO2022101510, U.S. Pat. No. 11,485,969 B2 and UY38805, among others. However, these devices require slow laboratory methods that prevent a timely and reliable response from the epizootiological point of view for decision-making to users of animal health and production.

Regarding predictive technologies or real-time risk identification, the company Boehringer Ingelheim Vetmedica GmbH has developed the “SoundTalks” technology, based on recordings of animal weights and their sounds. On the other hand, methods and computer systems have been developed to establish disease risk indicators such as those indicated in patent applications CN112785198, U.S. Patent Application Publication 2022136730, U.S. Patent Application Publication 2021358632 and CN112986503, which could provide fast and online information, however they do not detect specific pathogens or do so with low sensitivity and specificity, since they focus on establishing predictive models with environmental variables such as air pollutants or images and not on the health of the animal itself at the population level for a specific epidemiological unit.

It is therefore necessary to have an adequate health management model that meets the maximum efficiency in all its steps to comply with the good care of the corresponding animal populations and that allows anticipation through early detection of infections in animals and the prevention of the spread of infectious diseases to ensure food safety and protect animal and human health.

The present invention describes a method that includes processes to measure the environmental infection pressure from the concentration of a pathogen in the air, in a similar way to what has been used to detect airborne transmission of SARS-CoV-2 (Krieguel et al., 2022), with the difference of establishing disease alerts in production animal epidemiological units for any pathogen with appropriate predictive statistical methods.

The present invention establishes a baseline on the pressure of infection, which makes it possible to anticipate the appearance of a disease and thus generate alerts in epidemiological units of production animal populations. Environmental Pressure of Infection is understood as the determination of the amount of pathogenic microorganisms present in the air, for a given space-time, and their ability to infect a population.

It is worth mentioning that although the concept “Pressure of Infection” exists in the state of the art (Perea Gayosso, 2020), it is not applied at the population level from aerial samples as it is in the present application.

### SUMMARY

The present invention describes a method for generating alerts from the detection of the Environmental Pressure of Infection in epidemiological units of production animals. The method involves the determination of a baseline of Environmental Pressure of Infection from the collection of environmental samples, nucleic acid extraction, and molecular analysis, which is optimized using epizootiological and statistical techniques.

### BRIEF DESCRIPTION OF THE DRAWINGS

FIG. 1 is a comparative table of the method of the present invention vs. ELISA.

FIG. 2 is an image of the environmental sampling result vs. cloacal sample.

FIG. 3 is an image of environmental avian bronchitis virus detection result vs. cloacal sample.

FIG. 4 is an image of environmental avian bronchitis virus detection result vs. ELISA.

FIG. 5 is an image of the sampling module Analysis-Count of viable microorganisms.

FIG. 6 is a table of results of Detection Limits in hermetic chamber.

FIG. 7 is a picture of DNA quantification results and different storage conditions.

FIG. 8 is a table of weekly results per shed in an Epidemiological Unit.

FIG. 9 is a table of results by phases in poultry production.

FIG. 10 is a construction image of Early Warning of Risk: Avian Bronchitis and Mycoplasma.

FIG. 11 is an image of the construction of the Environmental Baseline in the Hatchery.

FIG. 12 is an image of 16S metagenomics results in environmental samples from different phases of pig production.

FIG. 13 is an image of the Tree of Indicators and Reports.

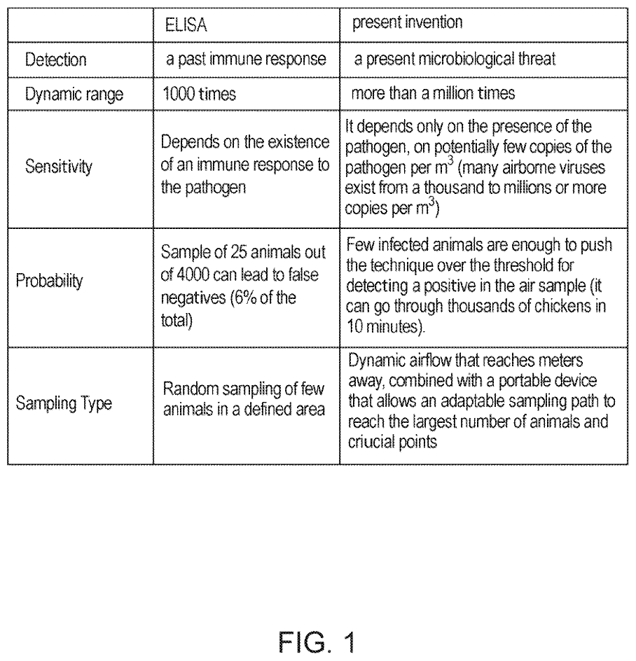

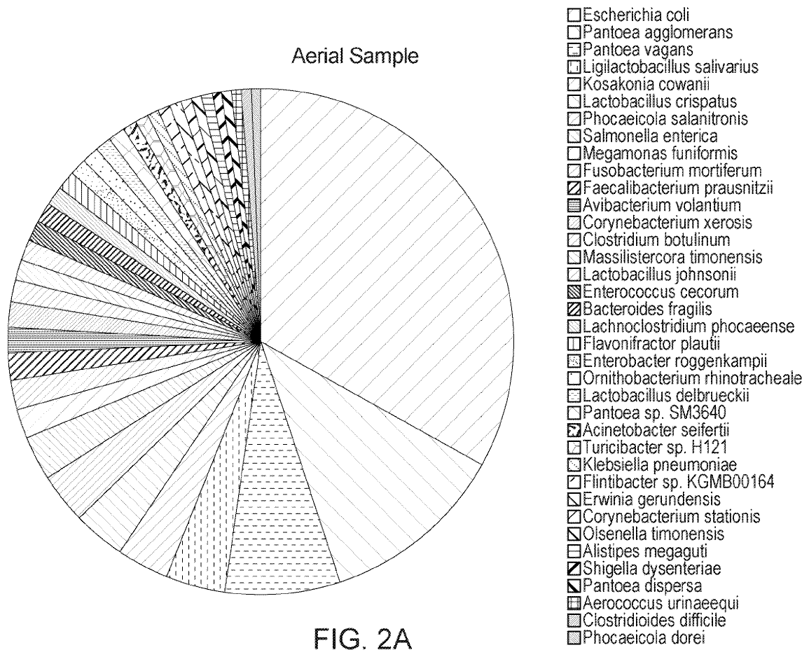

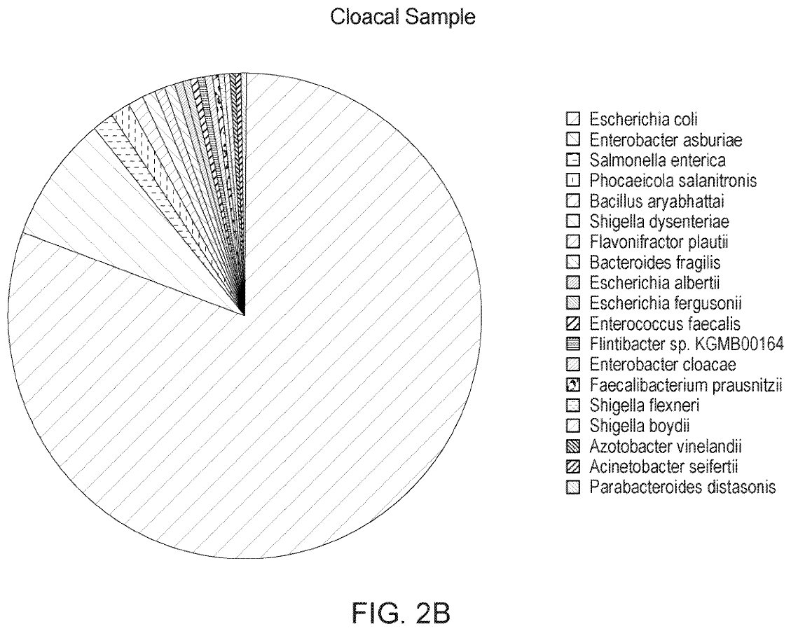

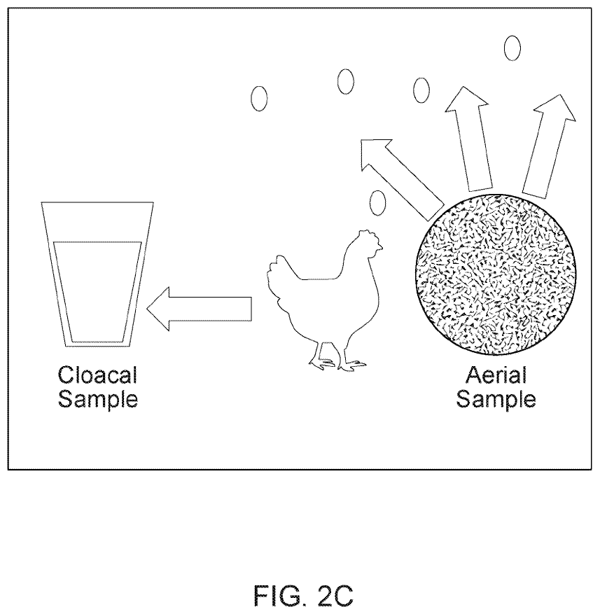

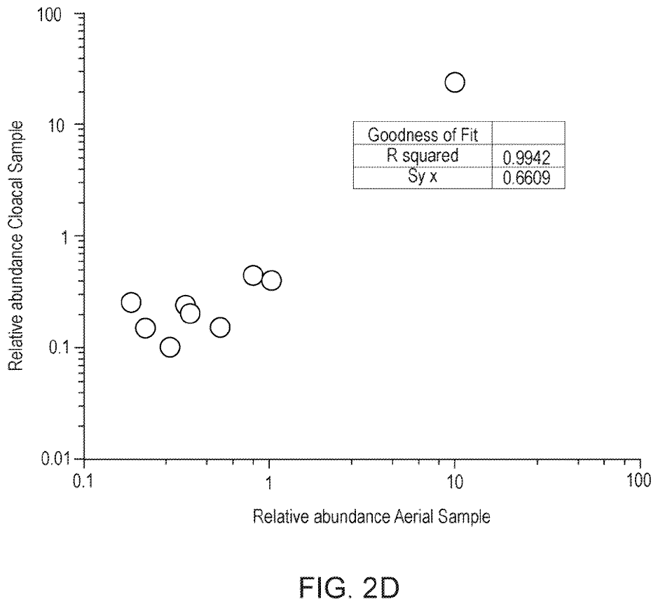

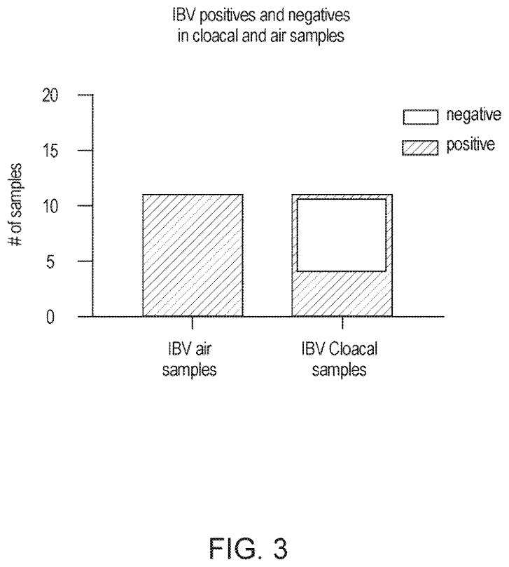

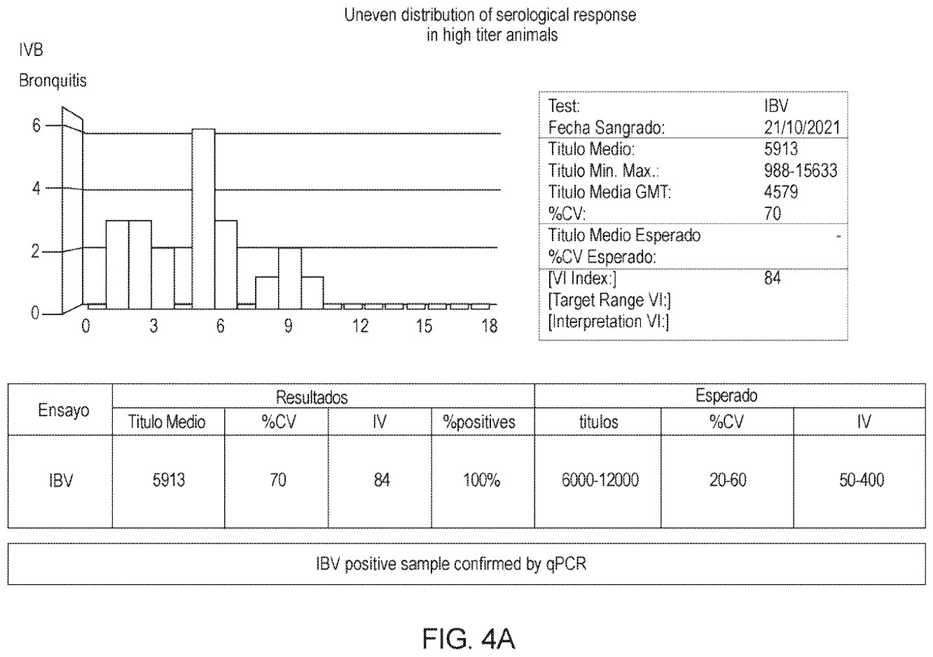

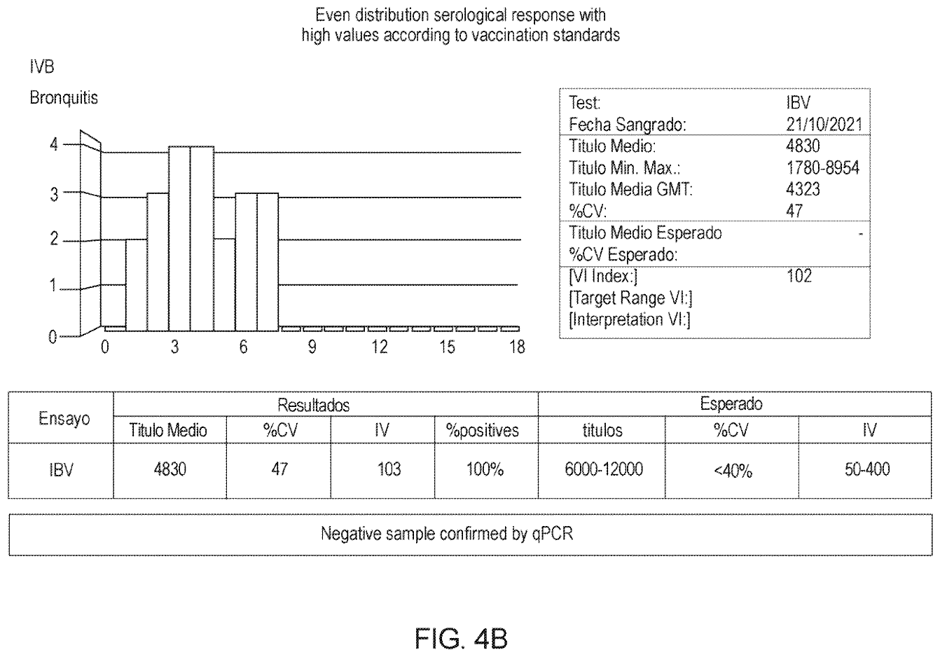

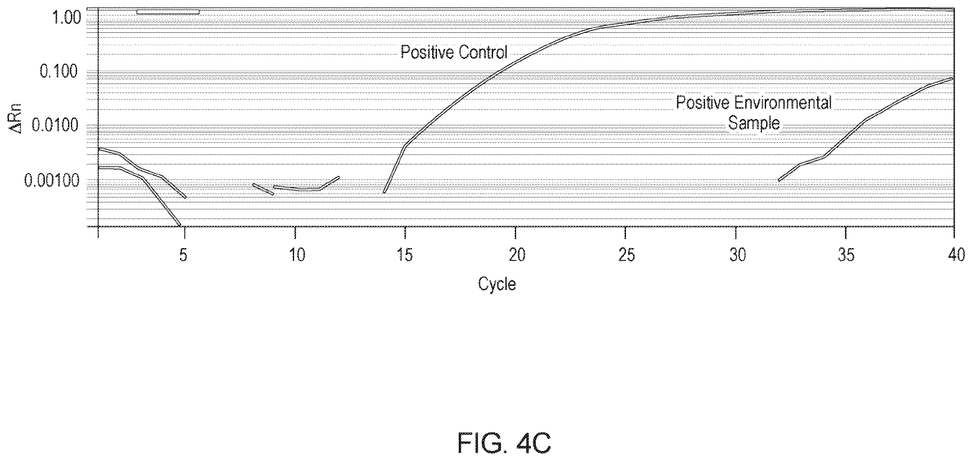

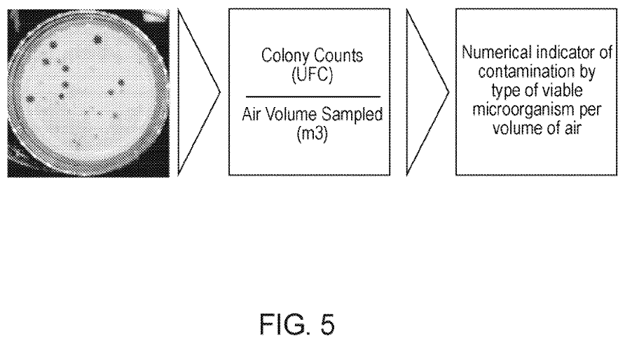

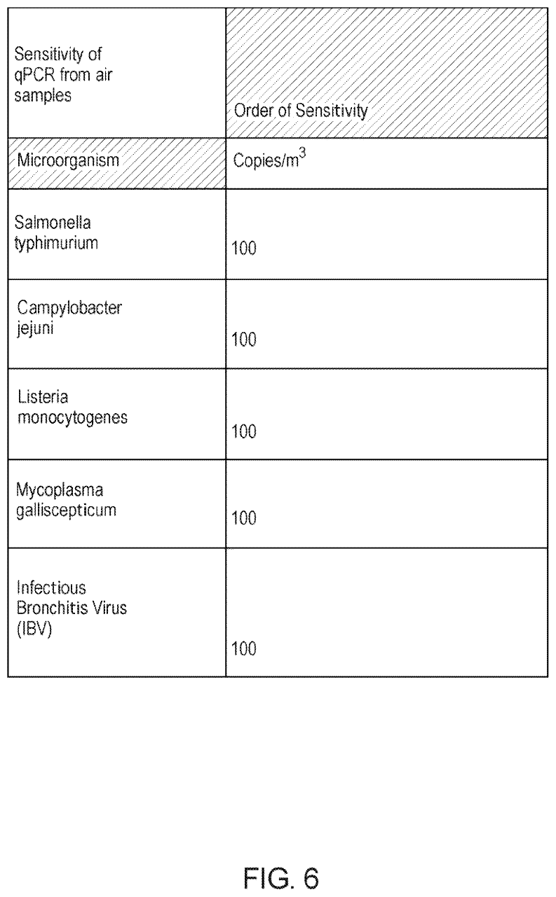

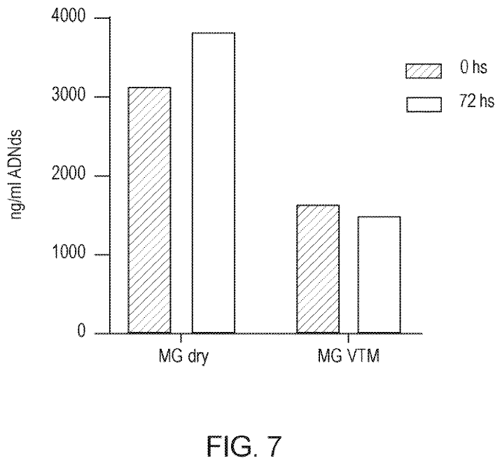

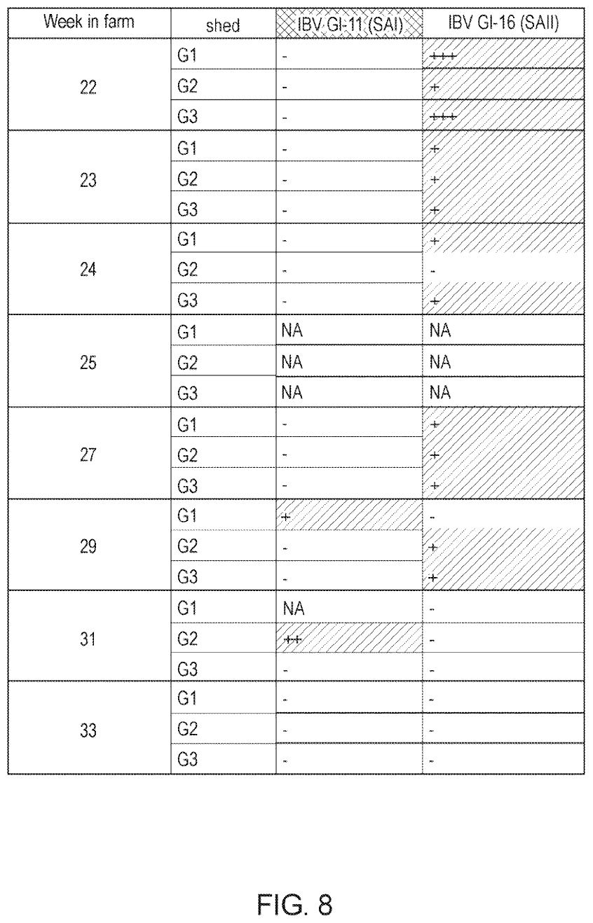

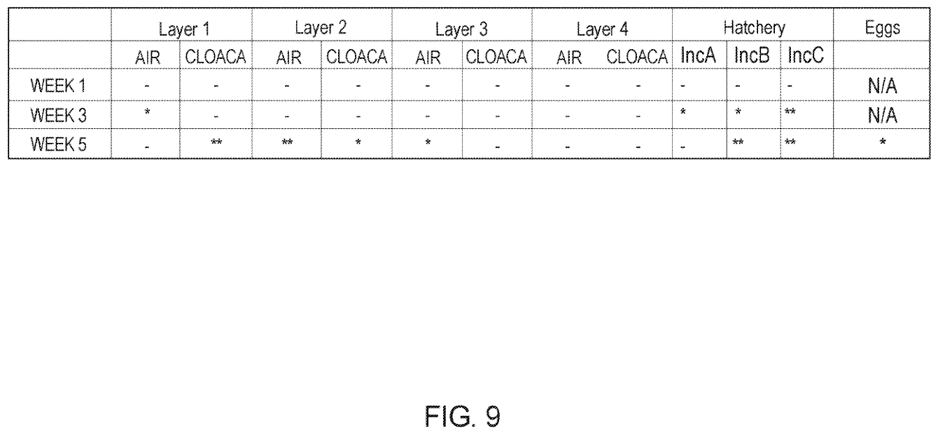

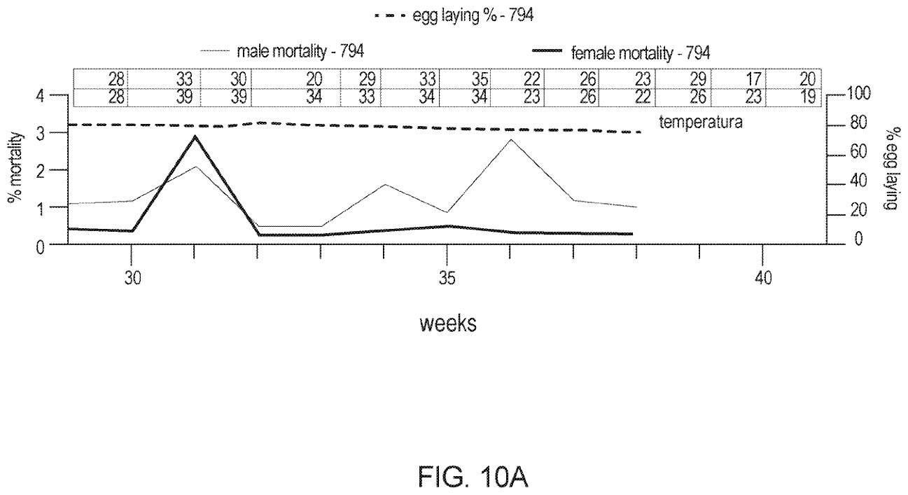

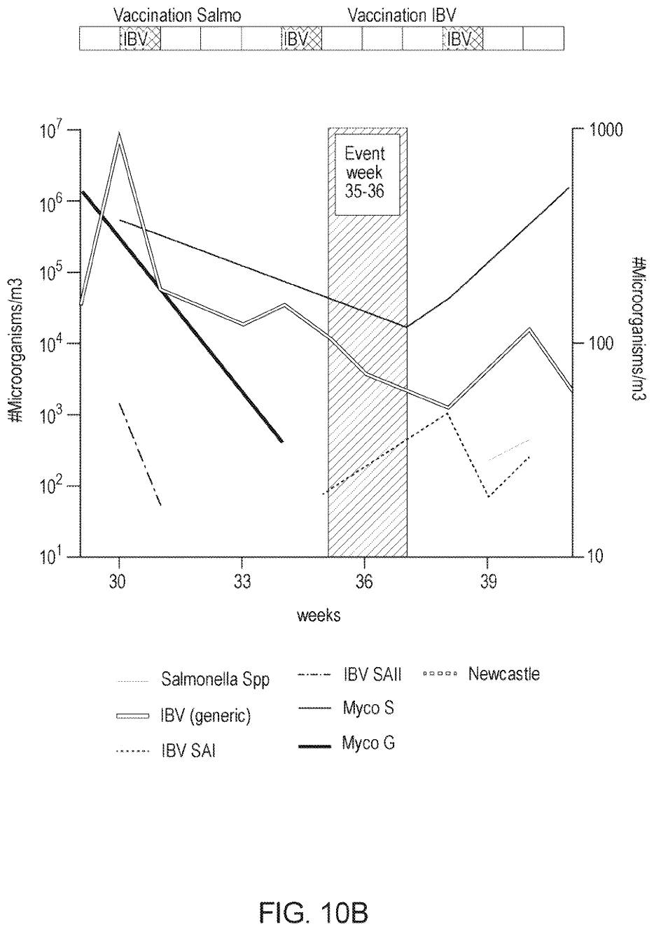

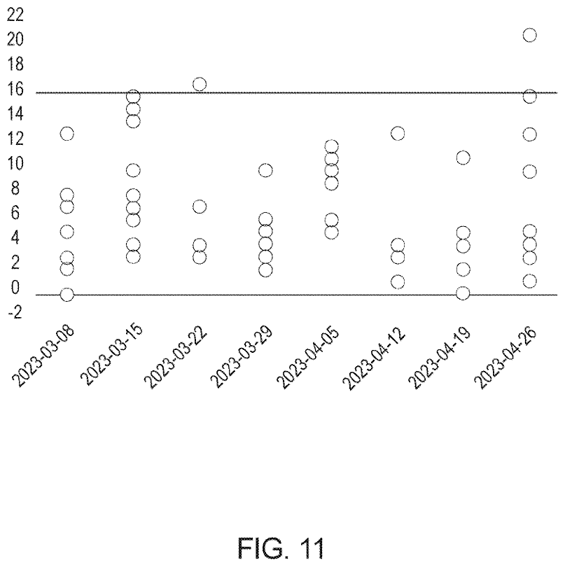

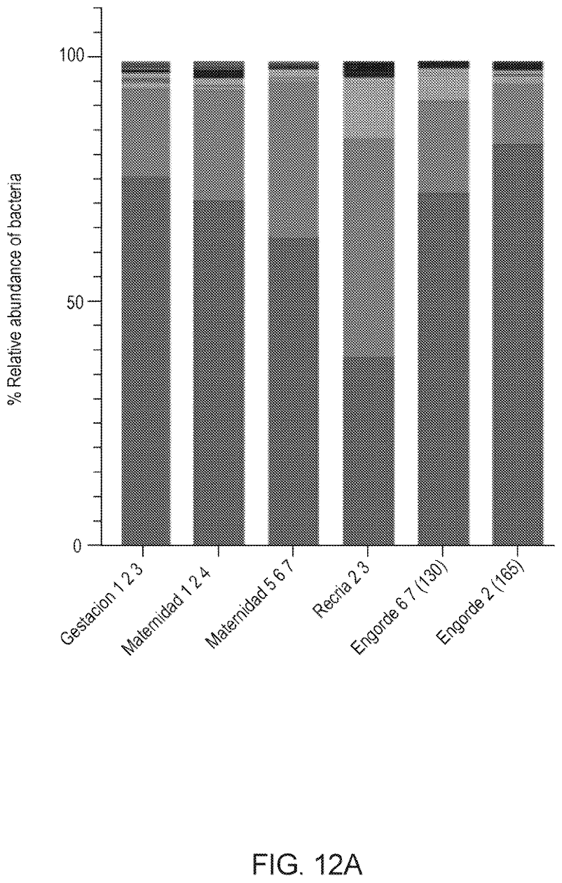

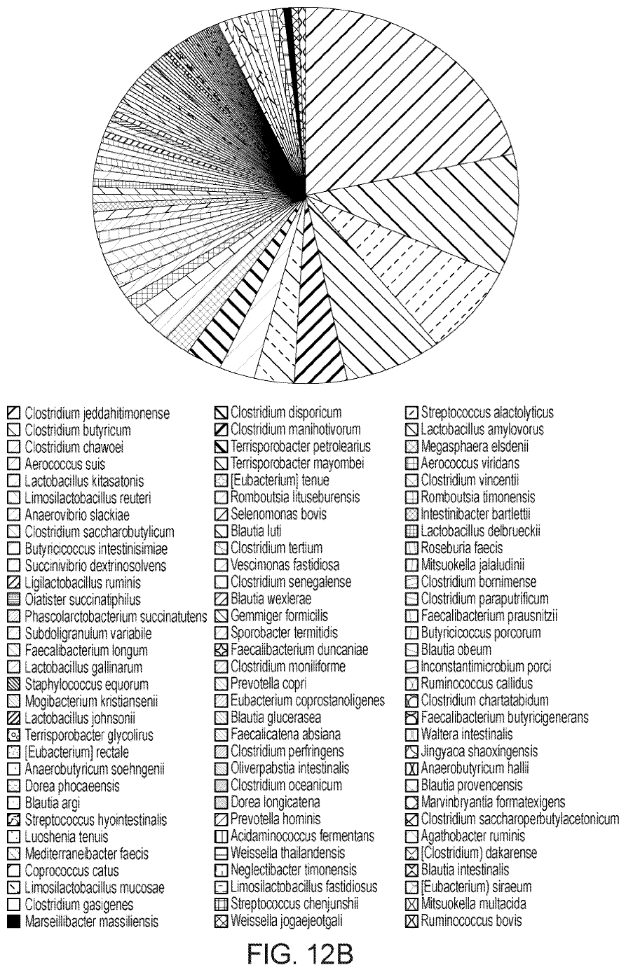

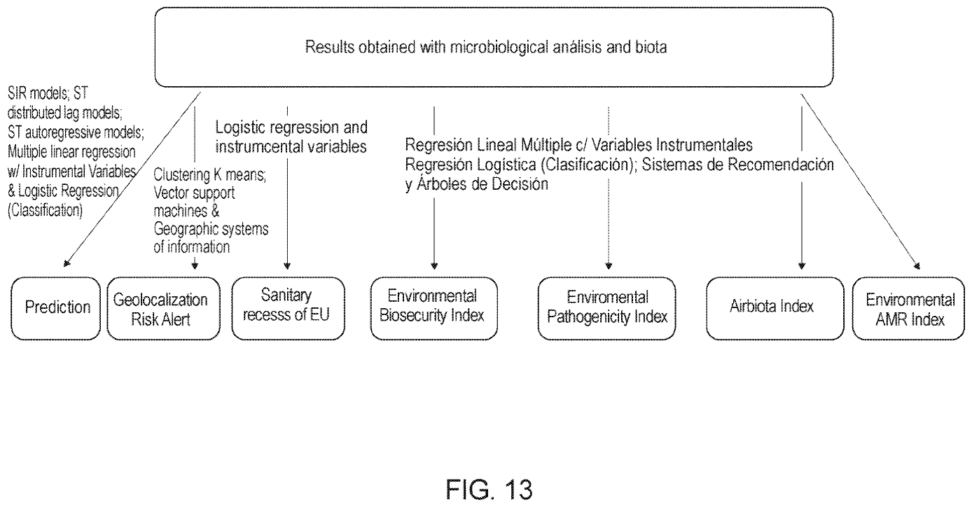

### DETAILED DESCRIPTION OF THE INVENTION

### Definitions

“Environmental Pressure of Infection” (EPI in its English acronym) as used herein refers to the concentration of a pathogen at a given time in the environment. This is expressed in units of concentration X.

“Threshold Environmental Pressure of Infection” (EPIt in its English acronym) as used herein refers to the environmental concentration of a pathogen above which it can cause an infection/condition in animals. EPIt is also an empirically established Environmental Baseline.

The “Environmental Baseline” (EBL in its English acronym) is a graph of the Environmental Pressure of Infection over time for a given pathogen in a given environment.

The “over abnormal data”: are understood as all those values that are far enough from the typical data of the distribution of the variable under study, so that it implies an alert of some kind. Be it abnormal levels of high mortality, abnormal levels of low weight, abnormal levels of pathogen concentration, among others.

The term “CAPTUS” herein refers to the device for taking air samples according to UY38805.

The term “Epidemiological Unit” in this document refers to one or more animal production facilities containing the same lots of animals with the same sanitary management, feeding, safety measures, etc.

The term “User” in this document corresponds to a person or institution that makes use of the technology presented in this document.

The term “Epizootiology” as used herein refers to a branch of epidemiology that focuses on the study of diseases in animals, specifically in animal populations. It is responsible for investigating the distribution, risk factors, spread patterns and impact of diseases in animals. Epizootiology also analyzes the interactions between pathogens, host animals, and the environment, with the goal of better understanding animal diseases and developing appropriate prevention and control strategies.

The term “Sample” in this document refers to one or more filters or membranes of controlled pore size containing dust particles, microorganisms, traces of genetic material, etc., which are obtained as a result of a filtration process and are representative of an environment. A sample must have information about the environment to which it corresponds, such as sampling time and duration, the nomination of the sampled environments, a location, an associated production phase, a batch (or similar), a user, and other identifiers of the sampling process that makes it unique.

The term “Production Phase” in this document refers to one of the stages of the production process of animal husbandry that can be differentiated, by the age of the animals, and/or the processes that are carried out with them.

The term “Prediction” in this document refers to the ability to provide a forecast of the sanitary/productive status, based on data analytic techniques.

The term “Sectorized” in this document refers to an item or sector in the sense of type of animal production, such as fattening pigs, chickens, or egg production, or incubators, etc.

The term “Animals in Confinement” in this document refers to those groups of animals (generally for commercial use) that are located in a shed or similar closed or semi-closed enclosure, during the production phases.

The term “Geolocation Risk Alert” in this document refers to an alert triggered by the detection of microbiological threats, the existence of an infectious outbreak or the presence of pathogens in one location that may affect another location, depending on factors such as: type of threat, route of transmission, level of affectation of the locality and distance between one locality and another.

### INTRODUCTION

The problem to be solved with the present invention is the need to have information—in a quick and reliable manner—that allows anticipating the appearance of diseases for epidemiological units of animals in production. The proposed solution describes a method that allows reliable and early warnings of the appearance of diseases associated with specific pathogens to be delivered in a timely manner to the users of production animals.

The proposed method consists of measuring the Environmental Pressure of Infection (EPI) to report alerts based on an Environmental Baseline (EBL). These alerts allow decision makers access to high-quality information to mitigate or completely avoid the impact of diseases through the implementation of effective, appropriate, and data-based prevention and management strategies to reduce the spread of diseases and to protect the animal and human health.

The method involves taking environmental samples, storage and transport of the sample, classical microbiology assays and/or extraction of nucleic acids and molecular analyzes (sequencing, PCR, LAMP and others).

It is worth mentioning that although the concept “Pressure of Infection” exists in the state of the art (Perea Gayosso, 2020), it is not applied at the population level from aerial samples as it is in the present application.

Although there are specific devices and methods to determine the presence of a pathogen in the environment, such as those indicated in patent applications WO2021250274A1, WO2022101510, U.S. Pat. No. 11,485,969B2, they do not produce alerts for the appearance of diseases as an output of the process.

Additionally, in the U.S. Patent Application Publication 2020131509A1, although a step-by-step method is presented for the extraction and sequencing of DNA/RNA to determine the biota present in the air, this method does not include all the steps and specifications of the present application, from sampling to reporting alerts resulting from the Environmental Pressure of Infection measurement method.

In turn, from the discipline of epizootiology, patent applications WO2012115601A1 and U.S. Patent Application Publication No. 2021293817A1, although they refer to the monitoring and analysis of infectious agents and epizootic risks, they do not include all the steps of current application nor do they include a stage for measuring Environmental Pressure of Infection, nor do they obtain alerts as a result as it is the case of the present application. In addition, they focus on measurements at the individual and non-population level (WO2012115601A1) and the monitoring is fixed instead of a mobile device like the one used in the present invention (UY38805).

The present invention allows to know the behavior of any pathogen in an environment of animal production, being able to compare, for example, the behavior by seasons, in a fast and precise way, having the possibility from the first day of implementation to receive alerts that allow preventive measures to be taken and corrective measures to reduce the impact of the disease on animal health.

### Method Description

The method to predict the risk of diseases in epidemiological units of production animals of current application is carried out with the establishment of an Environmental Baseline.

The Environmental Baseline is a tool that allows knowing, from the Environmental Pressure of Infection, the behavior of a given pathogen over time in a particular establishment or farm. It also allows establishing the critical concentration limits for said pathogen from which they mean a risk for production. Such critical limits arise from evaluating extreme outliers as those that lie outside the boundary. In this way, when a concentration at a given time, in a given farm or establishment, exceeds the established critical limits, alerts can be issued.

Specifically, the method follows the following steps: Determination of Environmental Pressure of Infection (EPI), Establishment of threshold Environmental Pressure of Infection (EPIt), Construction of the Environmental Baseline (EBL) and Generation of disease risk alerts.

1. Determine Environmental Pressure of Infection (EPI): Given a pathogen A from an epidemiological unit, proceed as follows in order to determine its specific concentration (X_{A}) in the environment:

Carrying out standardized sampling (a certain time and protocol) with air sampling equipment according to the type defined in patent application UY38805. Obtaining a filter (sample) containing dust particles with microorganisms and associated genetic material of said microorganisms.

Storage and transport of the sample at room temperature to the laboratory without the need to add stabilizing solution, which is stable of up to 72 hours.

The sample is subjected to a mixture of physical and chemical extractive processes that maximize the amount of genetic material extractable from the filter, which may include bead shaking, enzymatic digestion, centrifugation, and/or other validated/accessory extractive methods to ensure performance. A process consisting of these phases increases the sensitivity of subsequent analyzes from a small sample and ensures representativeness by minimizing bias and maximizing the chance of detecting diversity of pathogens in the sample. Typically, a sample of less than 100 ul containing DNA or RNA is obtained to distribute among a variety of assays. DNA/RNA quantification is performed using fluorometry and/or absorbance methods. The information obtained from the sample is nanograms/m³.

In one representation, samples are used to extract DNA or RNA to apply qPCR with fluorescent probes in simple or multiplex format in which a Ci value is obtained, which correlates with the number of copies of A in the farm/establishment. The information obtained from the sample is X_{A} expressed as copies/m³.

2. Establish threshold Environmental Pressure of Infection (EPIt): Given a pathogen A, the concentration (u_{A}) is established from which said pathogen can infect/affect animal health. The EPIt is specific to each pathogen and varies by epidemiological unit. It is built by carrying out field tests, that is, empirically, with the following method:

Selection of epidemiological units where there are close antecedents in time of suspected presence of A but that said epidemiological units are not, for example, in an endemic area, or there is suspicion of over-representation.

Selection of farms or establishments that have historical production data available (mortality, births, weight, posture, etc.), serological, and/or symptomatic.

Carrying out field tests on selected farms where the concentration (X_{A}) in air is measured weekly, for example, following the method explained in 1 (EPI).

Performance, during the field-testing period, of serological tests, and/or measurement of productive data (mortality, births, weight, posture, etc.) and/or identification of symptoms.

Realization through the transformation of attributes of X_{A} and of the productive data, to study the correlation by pairs (e.g. events of high mortality with events of high pathogen load X_{A}), resulting in a EPIt threshold of X_{A}=u_{A} that separates the levels that significantly affect production.

3. Environmental Baseline (EBL): Given an epidemiological unit in a farm or any productive establishment for a certain sector, or phase of the productive sector for which one wants to know; the Environmental Infection Pressure of a pathogen A, is measured over time (t), for example, with a weekly frequency, the concentration (X_{A}) in the air. The Environmental Baseline (EBL) will be characteristic and unique for each disease, and each establishment over time, being in constant feedback with new data.

The Environmental Baseline allows knowing the behavior of a given pathogen, giving early warnings, and is built as follows:

If there are no data of X_{A} at time (t) the EBL for the first sampling cycle is:
EBL₁=u_{A}, according to step 2 above.

For the second sampling cycle, the baseline will be:
EBL₂=Q3₂+C₁×RIC₂, where:

Q3₂ is the value that accumulates 75% of the ordered data in the second sampling cycle. RIC₂ is the interquartile range of the ordered data in the second sampling cycle and C₁=(u_{A}−Q3₁)/RIC₁ where u_{A} is the concentration of the pathogen determined in step 2. Q3₁ is the value that accumulates 75% of the ordered data in the first sampling cycle. RIC₁ is the interquartile range of the ordered data in the first sampling cycle.

For the third sampling cycle, the baseline will be:
EBL₃=Q3₃+((C₁+C₂)/2)×RIC₃, where: Q3₃ is the value that accumulates 75% of the ordered data in the third sampling cycle, RIC₃ is the interquartile range of the ordered data in the third sampling cycle and C₁=(u_{A}−Q3₁)/RIC₁, where u_{A} is the concentration of the pathogen determined in step 2, Q3₁ is the value that accumulates 75% of the ordered data in the first sampling cycle, RIC₁ is the interquartile range of the ordered data in the first sampling cycle C₂=(u_{A}−Q3₂)/RIC₂, where u_{A} is the concentration of the pathogen determined in step 2, Q3₂ is the value that accumulates 75% of the ordered data in the second sampling cycle, RIC₂ is the interquartile range of the ordered data in the second sampling cycle.

From the third cycle onwards, the baselines take the following form:
EBLᵢ=Q3ᵢ+Cᵢ×RICᵢ where:

Q3ᵢ is the value that accumulates 75% of the ordered data in the ith sampling cycle, RICᵢ is the interquartile range at the ith sampling cycle and Cᵢ is the reference coefficient in the ith sampling cycle, where:

$${C}_{i} = ( {C}_{1 +} {C}_{2 +} … {C}_{n - 1} ) / n - 1$$

In summary and expressed in an alternative way:

For sampling cycle 1, we use the reference:

$${EBL}_{1} = {u}_{A}$$

For each sampling cycle EBLₜ₊₁=Q3ₜ₊1+Cₜ×RICₜ₊₁ during the sampling cycle t+1 and Cₜ=(C₁₊C₂₊ . . . . Cₜ₋₁)/t−1.

At the end of each sampling cycle, a new coefficient Cₜ₊₁ is learned, which will be added to the average of the following sampling cycle:
Cₜ₊₁=(u_{A}−Q3ₜ₊1)/RICₜ₊₁ at the end of sampling t+1

4. The construction of an alert system is based on establishing EBLs or situations that require notifying the user, and some of these alerts are detailed below:

Early Risk Alert: It is a notification made to the user of an epidemiological unit that is triggered when a sample, for a pathogen A, exceeds the concentration established by EBL. For the first implementations this value is equal to EPIt, this means initially alerts are reported based on the known information of A, and not of X_{A}. This has the benefit of being able to issue alerts from the first day of implementation and then adapt these alerts to the reality of each epidemiological unit.

Geolocation Risk Alert: An alert is reported for being within a proximity range where an Early Risk Alert has been detected for a disease or pathogen that is highly relevant to the sector. This alert is built using georeferenced data to which a certain pathogen load or concentration is associated in the different locations. Said alert is carried out maintaining the anonymity of the exact origin of the pathogen.

A Universal Environmental Baseline (EBLᵤ) is built from all the data from farms or establishments of the same phase and productive sector. It is made up of the weighted average (by number of measurements) EBL, being EBLᵤ=i=1n(EBLi/n)/(Obsᵢ/Total Obs) where i is a given epidemiological unit, n is the total epidemiological units belonging to the same phases and productive sectors, and Obsᵢ, are the number of observations (measurements of X_{A}, and/or other variables) in the epidemiological unit i, and Total Obs are the number of total observations that are carried out in all the farms of that phase and productive sector. This EBL, being of a universal nature and adjusting with the data that enters as monitoring is carried out, is updated, evolves, and becomes more precise as a reference measure for the sector.

### EXAMPLES

### Working Example 1: Environmental Sampling Vs. Cloacal Sampling

As proof of the potential representativeness that the environmental sample can achieve as an indicator of processes that occur in animals, analytic tests of genetic diversity and correspondence of species shared between two paired types of sampling in broilers, one cloacal and the other of the aerial environment, are shown. DNA was extracted and subjected to 16s gene sequencing for diversity analysis. Both samples show shared species and particular species of each sample. The species of bacteria shared between the cloacal sample and the aerial sample in environments where broilers are raised reached an R2 of 0.6-0.9. This shows a high degree of representativeness in what the aerial sample tells us about the cloacal sample and validating the aerial approach for the environmental monitoring of the microbiological reality of the animals.

### Working Example 2: Detection of Environmental Vs Cloacal Avian Bronchitis Virus

In a monitoring trial for the presence of Avian Bronchitis Virus (IBV), which is a pathogen of preferential transmission by air, in poultry farms for different farms and times, it was seen that for positive air samples by qPCR, less than 50% of its cloacal counterpart was positive. This shows that, to assess the presence of preferentially airborne pathogens, sampling with CAPTUS and within this sampling scheme is superior to cloacal sampling, which can be more laborious, invasive, and ultimately less representative.

### Working Example 3: Detection of Avian Bronchitis Virus by Environmental qPCR Vs. Necropsy

In events of aerial environmental detection of pathogenic IBV strains, consistent with pathogenesis according to the veterinarian responsible for health of the establishment, samples from three tracheas of infected animals from the same farm were sent to the laboratory a week later to be analyzed with the same qPCR. None of the tracheas tested positive, showing that the weekly environmental monitoring method arrives on time to detect dynamics that, if post-symptom detection is attempted, it may be more difficult to identify the pathogen responsible for the cause.

### Working Example 4: Detection of Environmental Avian Bronchitis Virus Vs ELISA

In chicken farms, the correlation between the presence of pathogenic IBV (SAI and SAII strains) and the serological response of the birds was estimated. Farms were sampled for 6 weeks environmentally and blood sampling for serology by ELISA was performed at the end of the cycle. Farms positive for pathogenic IBV by qPCR had a serological profile with higher ELISA values and inhomogeneous dispersion, while IVB-negative farms had a cluster of ELISA values consistent with vaccination without IVB challenge. Additionally, it is shown what the amplification of the positive environmental sample looks like for this case, compared to a positive control. This demonstrates the correlation between infection events that can be detected weeks before and immunological effects, consistent with the infection history.

### Working Example 5: Analysis Sampling Module-Viable Microorganism Count

Aerial samples can be captured with CAPTUS, and the filters directly seeded on a plate with culture medium to analyze the growth of different types of microorganisms, such as aerobes, fungi, yeasts, enterobacteria, or more specific groups, allowing viable microorganisms to be counted. Through an analysis of an image, manual or automatic counts of the number of colonies that grow in a filter can be made after an adequate time (depending on the type of microorganism and their density in the sample). The example shows the environmental contamination of aerobic microorganisms that grows in a filter obtained after 10 minutes of sampling, which corresponds to 1m³ of air, the direct count of the filter in this case can be expressed as the number of aerobic microorganisms per m³, and it can be taken as a numerical indicator of the contamination of that environment.

### Working Example 6: Detection Limits in a Hermetic Chamber

To demonstrate the sensitivity of the method under controlled conditions, a variety of microorganisms of interest in the animal and food production industry were spread in an airtight chamber. Suspensions of microorganisms were volatilized using ultrasound, which generates airborne particles, and air samples of dispersed microorganisms were taken with CAPTUS for a standard time of 10 minutes. Various dilutions from millions to tens of microorganisms per m³ were tested. As a result, dilutions that had an order of 100 microorganisms per m³ (or higher) resulted in successful amplifications by specific qPCR for these pathogens/vaccines.

### Working Example 7: DNA Quantification Data and Different Storage Conditions

Regarding the extraction of DNA from air samples, the dry filter is superior to the filter extracted in viral transport medium (VTM), close to 3 times more efficient than the extraction process alone, and there is greater stability at 72 hours at room temperature. This shows that beyond the simplicity of dry storage, the method of transport and dry storage from the extraction itself is also superior.

It is also noteworthy that the qPCRs that are carried out with a dry filter compared to the MTV filter, have a better CT, being able to increase the detection sensitivity of RNA viruses such as the vaccinal IBV between 100 and 200 times.

### Working Example 8: Criterion to Make a Pool

An environmental monitoring was carried out and the monitoring of IBV in air samples allowed to observe a transfer from one shed to another, within the same Epidemiological Unit, in less than a week.

Environmental monitoring was carried out applying the method described in this invention on a weekly basis for IBV and other pathogens. This was carried out for three separate sheds, but which constituted the same epidemiological unit. Each house had 4,000 or more breeder hens. The samples were taken to the laboratory in search of non-vaccinal strains of IBV, by the qPCR method. For each case that infections with IBV field strains were detected, two or more infected houses were detected at a maximum distance of one week, by type of pathogen. This means that an infection starts where it starts, or it is first found in 2 or 3 sheds, or the following week it can be found in another shed different from the one originally found. This accounts for the transmissibility within the epidemiological management unit on the farm, and our ability to monitor the environment following the infection dynamics.

### Working Example 9: Traceability Between Environments/Phases

In this example, the correspondence between the appearance of Salmonella in air and the presence in the following phases of incubators and eggs is observed, where it is observed that what begins in the air on the farm, continues to incubators and ends in the eggs. 4 flocks of egg-laying hens were environmentally monitored (Aerial+cloacal) for the presence of Salmonella spp (and with subsequent Enteritidis/Typhimurium differentiation), the incubators were environmentally monitored (air) at those times and a control was also carried out on the surface of the eggs. This example shows how it went from a first week free of salmonella in the environment and cloaca, to a following sampling date with first evidence of salmonella in air on farms, which was accompanied by salmonella in air from hatcheries. Finally, presence is observed in more farms at an environmental level and presence in incubators, as well as on the surface of the eggs.

### Working Example 10: Anticipated Warning of Risk

The detection of viruses and bacteria that combine at a time of infection can give increased productive losses due to a coinfection/infection sequence that generates increased effects. In the following example, the continued presence of Mycoplasma synoviae (myco S) in this flock of breeding hens is added for the first time, for that farm, the detection of increasing levels of an IBV SAI strain. This coincidence of these two pathogens, which are known to be synergistic in pathogenicity, coincides with a higher detectable mortality peak at week 35-36, which does not correspond to heat peaks or other causes. At week 35, alerting of the presence of IBV-SAI in the presence of Mycoplasma synoviae, constitutes a management tool to avoid consequences of the joint infection peak (as can be seen, after the environmental detection of IBV-SAI Mycoplasma rises again and other pathogens such as salmonella begin to rise in recent weeks as well). In future instances, the producer administered antibiotics against secondary infections by recording the IBV-SAI detection peaks to avoid the impact of bacterial infections that have a greater impact in the presence of non-vaccinal BVI. This shows that this method of weekly environmental monitoring of pathogens serves to warn at least 1 week before the peak of deaths, the possible causes to generate better treatments such as antibiotics and palliatives.

### Working Example 11: Environmental Baseline in Hatchery

The airborne environmental data from counts make it possible to reconstruct the reality of the contamination levels of the facilities and generate a baseline.

In this example, data on aerobic organism counts (UFC/m³), monitored by weekly aerial sampling in a general hatchery environment, were analyzed. With these data, a scatter graph of points by sampling date was constructed with a critical limit baseline of 16 UFC/m³ (black line) based on: 3×RIC Upper border: Q3-3×RICS, with Q1 and Q3 being the 25% and 75% quartiles, respectively, and RIC the interquartile range. In this way, data monitoring is carried out that allowed the issuance of alarms in reports to producers that contamination levels could go beyond normal levels. In the following example only two weeks report outliers (above the line) within their distribution. It should be noted that, in this same monitoring, the “post-cleaning” data of the facilities had lower values and the monitoring and alarm system allowed adjustments to the cleaning schedule.

### Working Example 12: Longitudinal Traceability on a Single-Site Farm

In the following example, aerial environmental sampling of a pig farm was carried out, in 6 environments of the different production phases, including gestation, farrowing, rearing, and fattening. Each phase is physically separated from another by tens to hundreds of meters, but they correspond to a single connected management unit. Bacterial diversity in air at different taxonomic levels was examined by massive sequencing, and how much they shared between phase and phase. The different phases showed environmental diversities ranging from tens to hundreds of species, which were grouped into major classes, which accounted for more than 80% of the total relative abundance in all samples and were shared among all production phases. At the species level, the representation of the 10-20 most abundant species was shared between phase and phase, but the relative abundances and the order in the ranking varied. The level at which the bacteria were shared in this environmental analysis shows us a microbiological reality that is transversal to the different sites at the community level of the farm.

These metagenomic results, when compared with the database created for the different phases of pig rearing, allow to report the airbiota index for each phase.

### Working Example 13: Tree of Indicators and Reports

The following figure details different indicators used to create epizootiological reports of the type of alerts and indices that allow users to take preventive or corrective actions.

### REFERENCES

Hosseini, S., Vázquez-Villegas, P., Rito-Palomares, M., Martinez-Chapa, SO (2018). Ventajas, Desventajas y Modificaciones del ELISA Convencional. En: Ensayo inmunoabsorbente ligado a enzimas (ELISA). SpringerBriefs en Ciencias Aplicadas y Tecnología ( ). Springer, Singapur. https://doi.org/10.1007/978-981-10-6766-2_5.
Pardo Cobas, MV (noviembre de 2006). Compendio de Epidemiología de la Universidad Nacional Agraria. Accessed from https://repositorio.una.edu.ni/2439/1/nl73p226.pdf
Perea Gayosso, J. (2020, 7 de julio). Consideraciones sobre la presión de infección en granjas porcinas. Accessed from https://www.engormix.com/porcicultura/articulos/consideraciones-sobre-presion-infeccion-t45665.htm
Krieguel M. et al (Int. J. Environ. Res. Público Salud 2022, 19 (1), 220). Accsessed from https://www.mdpi.com/1660-4601/19/1/220.

1. A method for disease risk prediction in epidemiological units of production animals comprising:
a) determination of Environmental Pressure of Infection (EPI);
b) establishment of threshold Environmental Pressure of Infection (EPIt);
c) construction of Environmental Baseline (EBL); and
d) generation of disease risk alerts.

2. The method of claim 1, wherein the determination of the Environmental Pressure of Infection comprises determining a value X_{A} calculated for epidemiological units of production animals, where X is a concentration value and A is a pathogen value.

3. The method of claim 2, wherein the value X_{A} expresses the concentration of a pathogen measured as the number of copies of the pathogen per cubic meter.

4. The method of claim 2, wherein the value X_{A} value is obtained from application of qPCR on DNA or RNA extracted from air samples.

5. The method of claim 1 wherein the determination of the threshold Environmental Pressure of Infection (EPIt) comprises a value u_{A} calculated for epidemiological units of animals, where U is concentration value from which a pathogen can infect/affect the health of animals and A is a pathogen value.

6. The method of claim 5, wherein the value u_{A} value expresses threshold concentration of a pathogen measured as the number of copies of the pathogen per cubic meter.

7. The method of claim 5, wherein the value u_{A} is obtained from correlation between critical production values selected from mortality, weight, births, feed conversion to weights, to events of the pathogen X_{A} in representative epidemiological units.

8. The method of claim 5, wherein the value u_{A} is equivalent to a threshold at which the threshold Environmental Pressure of Infection (EPIt) of the concentration of a pathogen A affects animal production, wherein, EPIt of X_{A}=u_{A}.

9. The method of claim 1, wherein determination of Environmental Baseline (EBL) comprises determining a value X_{A} calculated for epidemiological units of production animals, where X is a concentration value and A is a pathogen value, over time (t) for a disease in an epidemiological unit of animals. specific, where if there is no data of X_{A} at time (t), then the Environmental Baseline is u_{A}.

10. The method of claim 1, wherein determination of the Environmental Baseline (EBL) comprises determining a value X_{A} calculated for epidemiological units of production animals, where X is a concentration value and A is a pathogen value, over time (t) for a disease in an epidemiological unit of animals, wherein there is no data for X_{A} at time (t) and EBL is u_{A}.

11. The method of claim 10, wherein for the second sampling cycle EBL₂=Q3₂+C₁×RIC₂, wherein Q3₂ is a value that accumulates 75% of ordered data in the second sampling cycle, RIC₂ is the interquartile range of the ordered data in the second sampling cycle and C₁=(u_{A}−Q3₁)/RIC₁ wherein u_{A} is a concentration of a pathogen determined, Q3₁ is a value that accumulates 75% of ordered data in the first sampling cycle and RIC₁ is an interquartile range of ordered data in the first sampling cycle.

12. The method of claim 11, wherein for the third sampling cycle, EBL₃=Q3₃+((C₁+C₂)/2)×RIC₃, where Q3₃ is a value that accumulates 75% of ordered data in the third sampling cycle, RIC₃ is an interquartile range of the ordered data in the third sampling cycle and C₁=(u_{A}−Q3₁)/RIC₁, where u_{A} is a concentration of the pathogen, Q3₁ is a value that accumulates 75% of the ordered data in the first sampling cycle, RIC₁ is an interquartile range of the ordered data in the first sampling cycle, C₂=(u_{A}−Q3₂)/RIC₂, where u_{A} is a concentration of the pathogen, Q3₁ is a value that accumulates 75% of ordered data in the second sampling cycle and RIC₁ is an interquartile range of ordered data in the second sampling cycle.

13. The method of claim 12 wherein for each sampling cycle after the third sampling cycle, EBLᵢ=Q3ᵢ+Cᵢ×RICᵢ, wherein Q3ᵢ is a value that accumulates 75% of the ordered data in the ith sampling cycle, IQRᵢ is an interquartile range at the ith sampling cycle and Cᵢ is the reference coefficient in the ith sampling cycle, wherein Cᵢ=(C₁₊C₂₊ . . . Cₙ₋₁)/n−1.

14. The method of claim 1, wherein generation of alerts comprises notifications when a sample of X_{A} has a value different from previously determined Environmental Baseline.

15. The method of claim 14, wherein generation of alerts according comprises alerts for users of one or more animal epidemiological units individually, aggregated, geolocated, stratified, temporal, clustered, universal, compared, and referential.
